(19) United States
(12) Patent Application Publication
(10) Pub. No.: US 2025/0076320 A1
(43) Pub. Date: Mar. 6, 2025

(54) METHOD AND PROGRAM FOR DETECTING ADMINISTERED DRUG, AND DEVICE THEREFOR

(71) Applicants: A&T CORPORATION, Fujisawa-shi (JP); NIHON UNIVERSITY, Tokyo (JP)

(72) Inventors: Masashi SANDA, Fujisawa-shi (JP); Haruna KAWAKAMI, Fujisawa-shi (JP); Tomohiro NAKAYAMA, Tokyo (JP); Yasuo OKUMURA, Tokyo (JP); Norihisa TAKEI, Tokyo (JP); Michiko YAMAKAWA, Tokyo (JP); Noriko KIYOHARA, Tokyo (JP)

(21) Appl. No.: 18/819,590

(22) Filed: Aug. 29, 2024

(30) Foreign Application Priority Data
Aug. 31, 2023 (JP) JP 2023-141578

(51) Int. Cl. G01N 33/86

(52) U.S. Cl. CPC G01N 33/86

(57) ABSTRACT

The present disclosure provides a method to determine whether or not a patient has received administration of a direct oral anticoagulant (DOAC). The present disclosure provides a method comprising allowing magnetic particles in a reagent to move after the addition of a sample to a dry reagent containing magnetic particles, monitoring the movement signal of the magnetic particles, analyzing the monitored movement signal of the magnetic particles, and comparing the results of analysis with a threshold, and a program and an apparatus used for such method.

### CROSS REFERENCE TO RELATED APPLICATIONS

The present application claims priority from Japanese patent application JP 2023-141578 filed on Aug. 31, 2023, the content of which is hereby incorporated by reference into this application.

### BACKGROUND

### Technical Field

The present disclosure relates to a method for detecting an administered drug and a program and an apparatus therefor.

### Background Art

Anticoagulants are used to prevent blood coagulation. For example, anticoagulants may be used for prevention of thromboembolism. A representative example of an anticoagulant is warfarin. Warfarin inhibits biosynthesis of a plurality of blood coagulation factors in the liver to prevent blood coagulation. Another example of an anticoagulant is a direct oral anticoagulant (DOAC). DOAC directly acts on particular components of the blood coagulation system cascade, such as thrombin and Factor Xa. Examples of DOAC include dabigatran, rivaroxaban, apixaban, edoxaban, and betrixaban.

In the event of bleeding, a patient receiving administration of a direct oral anticoagulant (DOAC) may encounter a difficulty in hemostasis because of the influence of DOAC. If the administration of DOAC is known, an appropriate reversal agent (or a neutralizer) may be used depending on the type of DOAC in order to overcome such difficulty. In case of medical emergency, however, the drug history of a patient is often unknown, and it is difficult to identify the cause of a difficulty in hemostasis. In addition, a neutralizer for warfarin is different from a neutralizer for DOAC.

In the past, detection of DOAC administration has been attempted based on the clot waveform of a blood sample (Masatoshi Wakui, the Journal of Japanese Society on Thrombosis and Hemostasis, 2022; 33 (1): 69-75). At present, however, such method of detection has not yet been put to practical use.

JP 2022-62969 A proposes analysis of DOAC and the like by clot waveform analysis. Since the autoanalyzer disclosed therein is a large-size apparatus used in the central laboratory, it would take approximately 30 minutes to obtain the results from the collected sample even if such method of analysis is actually performed. Accordingly, such method may be difficult to meet the need for medical emergency that requires rapid decision-making. While the first step of the algorithm of the disclosed method of determination includes prolonging of the clotting time, prolonging of the clotting time of many types of liquid reagents necessitates the use of DOAC at a concentration exceeding the therapeutic range. In practice, accordingly, it is believed to be impossible to detect DOAC except for special cases such as cases of overdose.

An example of a method for blood coagulation analysis is the prothrombin time (PT) test. The prothrombin time (PT) test reflects the mechanism of the extrinsic pathway, and the PT time is prolonged because of lowered activity of coagulation factors VII, X, V, II, and I. PT can be analyzed by adding a thromboplastin reagent containing a tissue factor and calcium to the plasma (e.g., citrated plasma) or the whole blood and then measuring the clotting time. While the prothrombin time (PT) can vary depending on reagents and analyzer, variations caused by the reagents or apparatuses can be normalized using prothrombin time-international normalized ratio (PT-INR).

An example of another method for blood coagulation analysis is the activated partial thromboplastin time (APTT) test. The activated partial thromboplastin time (APTT) test reflects the mechanism of the intrinsic pathway, and APTT time is prolonged because of lowered activity of coagulation factors XII, XI, IX, VIII, X, V, II, and I. APTT can be analyzed by adding phospholipids, an activator (e.g., silica, kaolin, or ellagic acid), and calcium to the plasma (e.g., citrated plasma) or the whole blood and then measuring the clotting time.

A practical method for analysis that enables detection of anticoagulants is needed.

### SUMMARY

The present disclosure provides a method and an apparatus that would solve at least part of the problems described above. More specifically, the present disclosure provides a method for identifying the type of anticoagulant that has been administered to a patient using a blood sample obtained from the patient and an apparatus used therefor.

The present inventors conducted concentrated studies in order to solve the technical problems described above. The present inventors measured a sample obtained from a patient receiving administration of DOAC using an apparatus and a dry reagent for measuring blood coagulation by analyzing the movement signal of the magnetic particles. As a result, the present inventors discovered that such reagent would react with DOAC at relatively low concentration and prolong the clotting time. The present inventors also analyzed the clot waveform of the sample obtained from a patient receiving administration of DOAC, and, as a consequence, discovered that level of attenuation would be milder than that of other anticoagulants, such as warfarin. On the basis thereof, the present inventors discovered that the clot waveform analysis of the sample obtained from a patient receiving administration of DOAC using the apparatus for measurement of blood coagulation and the dry reagent used therefor by analyzing the movement signal of the magnetic particles would enable analysis of DOAC at low concentration, which could not be detected using existing reagents, in particular, liquid reagents. The present inventors also found that whether or not DOAC has been administered could be rapidly determined in emergency settings using an apparatus capable of measuring the blood coagulation within a short period of time and usable for point-of-care (POC) testing. Such findings led to the completion of the present disclosure including such findings as an embodiment.

The present disclosure encompasses the embodiments described below.

[1]A test method for determining whether or not a direct oral anticoagulant (DOAC) has been administered to a patient, comprising:
(i) a step of adding a sample to a dry reagent containing magnetic particles for measuring prothrombin time or a dry reagent containing magnetic particles for measuring activated partial thromboplastin time;
(ii) a step of allowing the magnetic particles in the reagent to move after the addition of the sample, and monitoring the movement signal of the magnetic particles;
(iii) a step of analyzing the movement signal of the magnetic particles monitored in step (ii); and
(iv) a step of comparing the results of analysis performed in step (iii) with a threshold that enables a clear distinction between control 1 not receiving administration of DOAC (hereinafter, referred to as “no-DOAC control 1”) and control 2 receiving administration of DOAC (hereinafter, referred to as “DOAC control 2”).
   [2] The method according to Embodiment 1, wherein the threshold is the arithmetic mean of the inclination of the change in the movement signal of the magnetic particles of no-DOAC control 1 over time and the inclination of the change in the movement signal of the magnetic particles of DOAC control 2 over time, wherein the inclination of the change in the movement signal of the magnetic particles over time is the inclination of the clot waveform, which is determined by subtracting the movement level of the magnetic particles attenuated by a given percentage, selected from the group consisting of 3%, 4%, 5%, 6%, 7%, 8%, 9%, 10%, 11%, 12%, 13%, 14%, and 15%, relative to the maximal value of the movement level of the magnetic particles, from the maximal value of the movement level of the magnetic particles.
   [3]. The method according to Embodiment 1, wherein the threshold is the arithmetic mean of the minimal value of the differential value of the inclination of the change in the movement signal of the magnetic particles of no-DOAC control 1 over time and the minimal value of the differential value of the inclination of the change in the movement signal of the magnetic particles of DOAC control 2 over time, wherein the inclination of the change in the movement signal of the magnetic particles over time is the inclination of the clot waveform, which is determined by subtracting the movement level of the magnetic particles attenuated by a given percentage, selected from the group consisting of 3%, 4%, 5%, 6%, 7%, 8%, 9%, 10%, 11%, 12%, 13%, 14%, and 15%, relative to the maximal value of the movement level of the magnetic particles, from the maximal value of the movement level of the magnetic particles.
   [4] The method according to Embodiment 1, wherein the threshold is the arithmetic mean of the minimal value of the second-order differential value of the inclination of the change in the movement signal of the magnetic particles of no-DOAC control 1 over time and the minimal value of the second-order differential value of the inclination of the change in the movement signal of the magnetic particles of DOAC control 2 over time, wherein the inclination of the change in the movement signal of the magnetic particles over time is the inclination of the clot waveform, which is determined by subtracting the movement level of the magnetic particles attenuated by a given percentage, selected from the group consisting of 3%, 4%, 5%, 6%, 7%, 8%, 9%, 10%, 11%, 12%, 13%, 14%, and 15%, relative to the maximal value of the movement level of the magnetic particles, from the maximal value of the movement level of the magnetic particles.
   [5] The method according to Embodiment 1, wherein step (iv) comprises:
(iv-1) comparing the results of analysis performed in step (iii) with a first threshold that enables a clear distinction between no-DOAC control 1 and DOAC control 2; and
(iv-2) comparing the results of analysis performed in step (iii) with a second threshold that enables a clear distinction between control 3 receiving administration of an anticoagulant which is not DOAC (hereinafter, referred to as the “non-DOAC anticoagulant control 3”) and DOAC control 2.
   [6] The method according to Embodiment 5, wherein the threshold is the arithmetic mean of the inclination of the change in the movement signal of the magnetic particles of non-DOAC anticoagulant control 3 over time and the inclination of the change in the movement signal of the magnetic particles of DOAC control 2 over time, wherein the inclination of the change in the movement signal of the magnetic particles over time is the inclination of the clot waveform, which is determined by subtracting the movement level of the magnetic particles attenuated by a given percentage, selected from the group consisting of 3%, 4%, 5%, 6%, 7%, 8%, 9%, 10%, 11%, 12%, 13%, 14%, and 15%, relative to the maximal value of the movement level of the magnetic particles, from the maximal value of the movement level of the magnetic particles.
   [7] The method according to Embodiment 5, wherein the threshold is the arithmetic mean of the minimal value of the differential value of the inclination of the change in the movement signal of the magnetic particles of non-DOAC anticoagulant control 3 over time and the minimal value of the differential value of the inclination of the change in the movement signal of the magnetic particles of DOAC control 2 over time, wherein the inclination of the change in the movement signal of the magnetic particles over time is the inclination of the clot waveform, which is determined by subtracting the movement level of the magnetic particles attenuated by a given percentage, selected from the group consisting of 3%, 4%, 5%, 6%, 7%, 8%, 9%, 10%, 11%, 12%, 13%, 14%, and 15%, relative to the maximal value of the movement level of the magnetic particles, from the maximal value of the movement level of the magnetic particles.
   [8] The method according to Embodiment 5, wherein the threshold is the arithmetic mean of the minimal value of the second-order differential value of the inclination of the change in the movement signal of the magnetic particles of non-DOAC anticoagulant control 3 over time and the minimal value of the second-order differential value of the inclination of the change in the movement signal of the magnetic particles of DOAC control 2 over time, wherein the inclination of the change in the movement signal of the magnetic particles over time is the inclination of the clot waveform, which is determined by subtracting the movement level of the magnetic particles attenuated by a given percentage, selected from the group consisting of 3%, 4%, 5%, 6%, 7%, 8%, 9%, 10%, 11%, 12%, 13%, 14%, and 15%, relative to the maximal value of the movement level of the magnetic particles, from the maximal value of the movement level of the magnetic particles.
   [9] The method according to Embodiment 1, wherein step (iv) comprises:
(iv-1) comparing the results of analysis performed in step (iii) with a first threshold that enables a clear distinction between no-DOAC control 1 and DOAC control 2; and
(iv-2) comparing the results of analysis performed in step (iii) with a third threshold that enables a clear distinction between control 4 not receiving administration of an anticoagulant (hereinafter, referred to as the “no-anticoagulant control 4”) and DOAC control 2.
   [10] The method according to Embodiment 9, wherein the threshold is the arithmetic mean of the inclination of the change in the movement signal of the magnetic particles of no-anticoagulant control 4 over time and the inclination of the change in the movement signal of the magnetic particles of DOAC control 2 over time, wherein the inclination of the change in the movement signal of the magnetic particles over time is the inclination of the clot waveform, which is determined by subtracting the movement level of the magnetic particles attenuated by a given percentage, selected from the group consisting of 3%, 4%, 5%, 6%, 7%, 8%, 9%, 10%, 11%, 12%, 13%, 14%, and 15%, relative to the maximal value of the movement level of the magnetic particles, from the maximal value of the movement level of the magnetic particles.
   [11] The method according to Embodiment 9, wherein the threshold is the arithmetic mean of the minimal value of the differential value of the inclination of the change in the movement signal of the magnetic particles of no-anticoagulant control 4 over time and the minimal value of the differential value of the inclination of the change in the movement signal of the magnetic particles of DOAC control 2 over time, wherein the inclination of the change in the movement signal of the magnetic particles over time is the inclination of the clot waveform, which is determined by subtracting the movement level of the magnetic particles attenuated by a given percentage, selected from the group consisting of 3%, 4%, 5%, 6%, 7%, 8%, 9%, 10%, 11%, 12%, 13%, 14%, and 15%, relative to the maximal value of the movement level of the magnetic particles, from the maximal value of the movement level of the magnetic particles.
   [12] The method according to Embodiment 9, wherein the threshold is the arithmetic mean of the minimal value of the second-order differential value of the inclination of the change in the movement signal of the magnetic particles of no-anticoagulant control 4 over time and the minimal value of the second-order differential value of the inclination of the change in the movement signal of the magnetic particles of DOAC control 2 over time, wherein the inclination of the change in the movement signal of the magnetic particles over time is the inclination of the clot waveform, which is determined by subtracting the movement level of the magnetic particles attenuated by a given percentage, selected from the group consisting of 3%, 4%, 5%, 6%, 7%, 8%, 9%, 10%, 11%, 12%, 13%, 14%, and 15%, relative to the maximal value of the movement level of the magnetic particles, from the maximal value of the movement level of the magnetic particles.
   [13] The method according to Embodiment 1, wherein the threshold is a threshold that makes the inclination in the clot waveform reach −1000, wherein the inclination is determined by subtracting the movement level of the magnetic particles attenuated by 5%, relative to the maximal value of the movement level of the magnetic particles, from the maximal value of the movement level of the magnetic particles, when analysis is performed using a CG02N analyzer and using Drihemato PT reagent as a dry reagent containing magnetic particles for measuring the prothrombin time and when Drihemato PT reagent is supplemented with 25 μl of a citrated whole blood sample, or a threshold that corresponds thereto when analysis is performed using another apparatus and using another dry reagent.
   [14] The method according to Embodiment 13, which comprises a step of determining that a sample is derived from a patient not receiving administration of DOAC when the inclination of the change in the movement signal of the magnetic particles over time is higher (<−1000) than the threshold (−1000) or determining that a sample is derived from a patient receiving administration of DOAC when the inclination of the change in the movement signal of the magnetic particles over time is the threshold (−1000) or lower (>−1000) than the threshold (−1000),
wherein the inclination of the change in the movement signal of the magnetic particles over time is the inclination of the clot waveform, which is determined by subtracting the movement level of the magnetic particles attenuated by 5%, relative to the maximal value of the movement level of the magnetic particles, from the maximal value of the movement level of the magnetic particles.
   [15] The method according to Embodiment 13, wherein, when the sample is determined to be derived from a patient receiving administration of DOAC as a result of comparison with the threshold, the method further comprises:
a step of administering a reversal agent for DOAC or suggesting administration of a reversal agent for DOAC to the patient.
   [16] The method according to any one of Embodiments 1 to 15, wherein DOAC is selected from the group consisting of dabigatran, rivaroxaban, apixaban, edoxaban, and betrixaban.
   [17] A program for executing the method of any one of Embodiments 1 to 16.
   [18] An information recording medium comprising the program of Embodiment 17 recorded thereon.
   [19] An apparatus for testing an anticoagulant comprising the program of Embodiment 17 integrated therein or the information recording medium of Embodiment 18 stored therein.
   [20] The present disclosure allows a rapid determination as to whether or not a patient has received administration of an anticoagulant.

### BRIEF DESCRIPTION OF THE DRAWINGS

FIG. 1 shows a representative example of a reaction slide used for a dry reagent for measuring the prothrombin time.

FIG. 2 is a partially exploded diagram of the reaction slide shown in FIG. 1.

FIG. 3A shows a clot waveform of a sample not receiving administration of the direct oral anticoagulant.

FIG. 3B shows a clot waveform of each sample receiving administration of edoxaban.

FIG. 4 shows a clot waveform of each sample receiving administration of rivaroxaban.

### DETAILED DESCRIPTION

In some embodiments, the present disclosure provides a test method for determining whether or not a direct oral anticoagulant (DOAC) has been administered. In some embodiments, this method may be a test method automated by an apparatus. In some embodiments, this method does not involve physician's decision making. In some embodiments, this test method or automated test method may comprise:
(i) a step of adding a sample to a dry reagent containing magnetic particles for measuring the prothrombin time;
(ii) a step of allowing the magnetic particles in the reagent to move after the addition of the sample, and monitoring the movement signal of the magnetic particles;
(iii) a step of analyzing the movement signal of the magnetic particles monitored in step (ii); and
(iv) a step of comparing the results of analysis performed in step (iii) with the threshold that enables a clear distinction between no-DOAC control 1 and DOAC control 2. In some embodiments, a sample may be citrated plasma. In other embodiments, a sample may be citrated whole blood.

A dry reagent containing magnetic particles is used to allow the magnetic particles in the reagent to move after the addition of the sample, and monitor the movement signal of the magnetic particles. A dry reagent containing magnetic particles is used to measure the prothrombin time (PT) herein, unless otherwise specified. In some embodiments, the prothrombin time (PT) is the prothrombin time-international normalized ratio (PT-INR). INR is calculated by the following equation:

INR=(patient's PT/normal PT)^{ISI} [Equation 1]

wherein “ISI” is the international sensitivity index determined for each PT reagent.

Examples of dry reagents containing magnetic particles include the Drihemato PT reagent (A&T Corporation) and reagents having similar functions for measuring the prothrombin time. In some embodiments, a dry reagent containing magnetic particles comprises:
(i) tissue thromboplastin;
(ii) magnetic particles;
(iii) a calcium salt;
(iv) a dry reagent layer solubility improving agent;
(v) a dry reagent layer reinforcing material; and
(vi) a pH adjuster (pH buffer). In other embodiments, a dry reagent for measuring the prothrombin time may further contain a heparin neutralizer and/or a defoaming agent.

Examples of other dry reagents containing magnetic particles include the Drihemato APTT reagent (A&T Corporation) and reagents having similar functions for measuring the activated partial thromboplastin time. In some embodiments, a dry reagent containing magnetic particles comprises:
(i) a phospholipid:
(ii) an activator;
(iii) magnetic particles;
(iv) a calcium salt;
(v) a dry reagent layer solubility improving agent;
(vi) a dry reagent layer reinforcing material; and
(vii) a pH adjuster (pH buffer). In other embodiments, a dry reagent for measuring the prothrombin time may further contain a heparin neutralizer and/or a defoaming agent.

In the method of the present disclosure, the results of analysis performed in step (iii) can be compared with a threshold that enables a clear distinction between no-DOAC control 1 and DOAC control 2, which enables a determination as to whether the sample is derived from a patient receiving administration of a direct oral anticoagulant or a patient not receiving administration of a direct oral anticoagulant. In some embodiments, this method does not involve the decision of a physician. In other embodiments, a physician may make the decision.

In some embodiments, the threshold can be the arithmetic mean of the inclination of the change in the movement signal of the magnetic particles of no-DOAC control 1 over time and the inclination of the change in the movement signal of the magnetic particles of DOAC control 2 over time. In some other embodiments, the threshold can be the arithmetic mean of the minimal value of the differential value of the inclination of the change in the movement signal of the magnetic particles of no-DOAC control 1 over time and the minimal value of the differential value of the inclination of the change in the movement signal of the magnetic particles of DOAC control 2 over time. In some other embodiments, the threshold can be the arithmetic mean of the minimal value of the second-order differential value of the inclination of the change in the movement signal of the magnetic particles of no-DOAC control 1 over time and the minimal value of the second-order differential value of the inclination of the change in the movement signal of the magnetic particles of DOAC control 2 over time. In some embodiments, the inclination of the change in the movement signal of the magnetic particles over time can be the inclination of the clot waveform, which is determined by subtracting the movement level of the magnetic particles attenuated by a given percentage, relative to the maximal value of the movement level of the magnetic particles, from the maximal value of the movement level of the magnetic particles. In some embodiments, the given percentage attenuated can be, for example, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50%.

In some embodiments, step (iv) can comprise:
(iv-1) comparing the results of analysis performed in step (iii) with a first threshold that enables a clear distinction between no-DOAC control 1 and DOAC control 2; and
(iv-2) comparing the results of analysis performed in step (iii) with a second threshold that enables a clear distinction between non-DOAC anticoagulant control 3 and DOAC control 2. This enables a determination as to whether or not the patient has received administration of DOAC and whether or not the patient has received administration of an anticoagulant that is not DOAC. Examples of anticoagulants that are not DOAC include, but are not limited to, warfarin, heparin (may be denoted as “unfractionated heparin”), low-molecular-weight heparin, danaparoid, fondaparinux, and argatroban.

In some embodiments, the second threshold can be the arithmetic mean of the inclination of the change in the movement signal of the magnetic particles of non-DOAC anticoagulant control 3 over time and the inclination of the change in the movement signal of the magnetic particles of DOAC control 2 over time. In some other embodiments, the second threshold can be the arithmetic mean of the minimal value of the differential value of the inclination of the change in the movement signal of the magnetic particles of non-DOAC anticoagulant control 3 over time and the minimal value of the differential value of the inclination of the change in the movement signal of the magnetic particles of DOAC control 2 over time. In some other embodiments, the second threshold can be the arithmetic mean of the minimal value of the second-order differential value of the inclination of the change in the movement signal of the magnetic particles of non-DOAC anticoagulant control 3 over time and the minimal value of the second-order differential value of the inclination of the change in the movement signal of the magnetic particles of DOAC control 2 over time. The inclination of the change in the movement signal of the magnetic particles over time can be the inclination of the clot waveform, which is determined by subtracting the movement level of the magnetic particles attenuated by a given percentage, relative to the maximal value of the movement level of the magnetic particles, from the maximal value of the movement level of the magnetic particles.

In some embodiments, step (iv) can comprise:
(iv-1) comparing the results of analysis performed in step (iii) with a first threshold that enables a clear distinction between no-DOAC control 1 and DOAC control 2; and
(iv-2) comparing the results of analysis performed in step (iii) with a third threshold that enables a clear distinction between no-anticoagulant control 4 and DOAC control 2. This enables a determination not only as to whether or not the patient has received administration of DOAC but also as to whether or not the patient has not received administration of an anticoagulant.

In some embodiments, the third threshold can be the arithmetic mean of the inclination of the change in the movement signal of the magnetic particles of no-anticoagulant control 4 over time and the inclination of the change in the movement signal of the magnetic particles of DOAC control 2 over time. In some other embodiments, the third threshold can be the arithmetic mean of the minimal value of the differential value of the inclination of the change in the movement signal of the magnetic particles of no-anticoagulant control 4 over time and the minimal value of the differential value of the inclination of the change in the movement signal of the magnetic particles of DOAC control 2 over time. In some other embodiments, the third threshold can be the arithmetic mean of the minimal value of the second-order differential value of the inclination of the change in the movement signal of the magnetic particles of no-anticoagulant control 4 over time and the minimal value of the second-order differential value of the inclination of the change in the movement signal of the magnetic particles of DOAC control 2 over time. The inclination of the change in the movement signal of the magnetic particles over time can be the inclination of the clot waveform, which is determined by subtracting the movement level of the magnetic particles attenuated by a given percentage, relative to the maximal value of the movement level of the magnetic particles, from the maximal value of the movement level of the magnetic particles.

The given percentage adopted to determine the inclination of the change in the movement signal of the magnetic particles over time (or the differential value or second-order differential value thereof) is not limited to 5%. For example, a given percentage can be selected from the group consisting of 3%, 4%, 5%, 6%,7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50%.

In some embodiments, no-DOAC control 1 can be derived from a no-DOAC-administered control group composed of the number of samples “n”, which is 5 or more, 10 or more, 20 or more, 30 or more, 40 or more, 50 or more, or 100 or more, for example, 1000 or more. In some embodiments, DOAC control 2 can be derived from a DOAC-administered control group composed of the number of samples “n”, which is 5 or more, 10 or more, 20 or more, 30 or more, 40 or more, 50 or more, or 100 or more, for example 1000 or more. In some embodiments, non-DOAC anticoagulant control 3 can be derived from a control group receiving administration of a non-DOAC anticoagulant composed of the number of samples “n”, which is 5 or more, 10 or more, 20 or more, 30 or more, 40 or more, 50 or more, or 100 or more, for example 1000 or more. In some embodiments, no-anticoagulant control 4 can be derived from a control group not receiving administration of an anticoagulant composed of the number of samples “n”, which is 5 or more, 10 or more, 20 or more, 30 or more, 40 or more, 50 or more, or 100 or more, for example 1000 or more.

In some embodiments, the threshold can be a threshold that makes the inclination in the clot waveform reach −1000, wherein the inclination is determined by subtracting the movement level of the magnetic particles attenuated by 5%, relative to the maximal value of the movement level of the magnetic particles, from the maximal value of the movement level of the magnetic particles when analysis is performed using the CG02N analyzer (A&T Corporation) and the Drihemato PT reagent (A&T Corporation) by adding 25 M 1 of a citrated whole blood sample. The apparatus and the reagent used in the method of the present disclosure are not limited to those indicated above. The method of the present disclosure can be implemented in the same manner when another apparatus and another dry reagent for measuring the prothrombin time are used. When analysis is performed using another apparatus and another dry reagent, the threshold for such apparatus and reagent can be the corresponding threshold that makes the inclination in the clot waveform reach −1000, wherein the inclination is determined by subtracting the movement level of the magnetic particles attenuated by 5%, relative to the maximal value of the movement level of the magnetic particles, from the maximal value of the movement level of the magnetic particles when analysis is performed using the CG02N analyzer (A&T Corporation) and the Drihemato PT reagent (A&T Corporation) by adding 25 μl of a citrated whole blood sample. A person skilled in the art would be able to determine a threshold that corresponds to the threshold that makes the inclination reach −1000 by conducting a routine verification test on the basis of the teaching of the present disclosure.

In some embodiments, a sample can be determined to be derived from a patient not receiving administration of DOAC when the inclination of the change in the movement signal of the magnetic particles over time is higher (<−1000) than the threshold (−1000), and a sample can be determined to be derived from a patient receiving administration of DOAC when the inclination of the change in the movement signal of the magnetic particles over time is the threshold (−1000) or lower (>−1000) than the threshold (−1000). The same applies to corresponding thresholds.

In some embodiments, when the sample is determined to be derived from a patient receiving administration of DOAC as a result of comparison with the threshold, a reversal agent for DOAC may be administered to the patient, or administration of a reversal agent may be suggested. In some embodiments, suggestion of administration is performed by an automated apparatus or system. In some embodiments, suggestion of administration does not involve actions of a physician.

In some embodiments, DOAC may be selected from the group consisting of dabigatran, rivaroxaban, apixaban, edoxaban, and betrixaban.

In some embodiments, a program, software, or algorithm for executing the method of the present disclosure is provided. In some other embodiments, an information recording medium comprising the program, software, or algorithm recorded thereon is provided. In some other embodiments, a coagulation analyzer comprising the program integrated therein or the information recording medium stored therein is provided. In some embodiments, the coagulation analyzer can be CG02N (A&T Corporation) or an apparatus having functions similar to those thereof. With the use of the CG02N analyzer, a combination of an oscillating magnetic field and a static permanent magnetic field is applied to the reagent supplemented with the sample at intervals of 0.5 seconds, and the movement signal of the magnetic particles are monitored at the same intervals (see, for example, the product brochure or instructions of CG02N (A&T Corporation)).

For the sake of convenience, the present disclosure will provide explanations in connection with apparatus CG02N (A&T Corporation). When measurement is initiated, apparatus CG02N turns electromagnets at the bottom of the reaction cell on and off, and allows magnetic particles to move in the reaction cell. CG02N observes the movement of the magnetic particles as the amount of change in the scattered light, and analyzes the change with the elapse of time to determine clotting time. When the CG02N analyzer is used, the change in the movement signal of the magnetic particles over time is inversely correlated to the change in the viscosity of the dry reagent. As the clotting reaction advances, the viscosity in the reaction cell increases, and the movement of the magnetic particles becomes slower. The clot waveform is analyzed by determining a difference between the waveform value (scattered light) after a given period of time from the point when an electromagnet is turned on and the waveform value (scattered light) after a given period of time from the point when electromagnet is turned off. Differences in the waveform values are plotted with the elapse of time, and a point at which the waveform value is lowered by a given percentage from the maximal value (also referred to as “peak TOP”) is designated as the end point. The period from the peak TOP to the end point is measured to obtain the measured value (seconds). The term “clotting time” used herein refers to the period from the peak TOP to the end point. It should be noted that the clotting time is designated for the purpose of convenience and that the clotting reaction may occur at a point before the maximal value of the movement level of the magnetic particles (peak TOP) is observed. On the basis of the clot waveform data obtained, (1) the maximal value of the movement level of the magnetic particles, (2) the time at which the maximal value of the movement level of the magnetic particles is obtained, (3) the movement level of the magnetic particles when the movement level of the magnetic particles is attenuated by 5% relative to the maximal level, and (4) the time at which the movement level of the magnetic particles is attenuated by 5% relative to the maximal level can be determined as feature amounts of the clot waveform. On the basis of feature amounts (1) to (4), the inclination of the clot waveform from the maximal value of the movement level of the magnetic particles to the movement level of the magnetic particles attenuated by a given percentage, selected from the group consisting of 3%, 4%, 5%, 6%, 7%, 8%, 9%, 10%, 11%, 12%, 13%, 14%, and 15%, such as 5%, relative to the maximal value of the movement level of the magnetic particles can be obtained.

In an exemplary method for preparing the dry reagent for measuring the prothrombin time, a buffer containing a calcium salt, an amino acid or salt thereof, or a saccharide is first prepared, highly active tissue thromboplastin is dissolved in the buffer, magnetic particles is added to the solution to prepare a final solution, a given amount of the final solution is dispensed onto a reaction slide, and the solution is frozen and lyophilized. The buffer may further contain a heparin neutralizer and/or a defoaming agent. A tissue thromboplastin solution may be prepared by dissolving a lyophilized product containing, for example, rabbit-brain-derived tissue thromboplastin or rabbit-brain-derived phospholipid in a purified water.

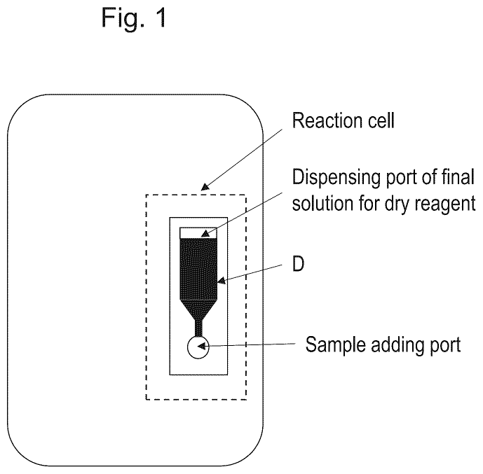

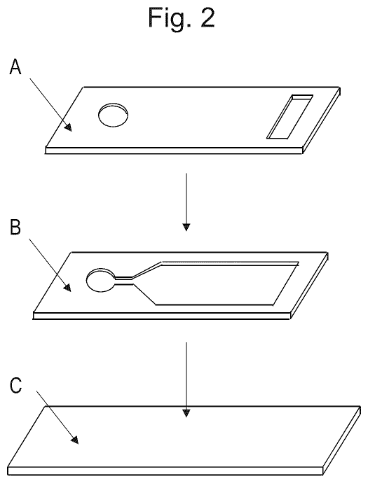

The reaction slide used in the method for preparation is not particularly limited, provided that, with the reaction slide, an increase in viscosity in the dry reagent for measuring the prothrombin time at the time of measurement of the prothrombin time can be optically monitored as an attenuation in the movement signal of the magnetic particles. Examples include a reaction slide shown in FIGS. 1 and 2. FIG. 1 shows a top view of the reaction slide. In FIG. 1, an area surrounded by a dotted line is a reaction cell composed of a dispensing port for the final solution for preparing the dry reagent for measuring the prothrombin time and a sample adding port. FIG. 2 shows the structure of a reaction cell in detail. The reaction cell is constructed by first applying a transparent polyester plate B to a white polyester plate C and then applying a transparent polyester plate A to the transparent polyester plate B. First, a surfactant solution is introduced into the reaction cell through the dispensing port shown in FIG. 1 and then suction-removed to hydrophilize the region D. Then, the final solution for the dry reagent for measuring the prothrombin time is injected into the reaction cell through the dispensing port to fill the region D with the final solution. When this type of reaction slide is used, in general, 20 to 30 μl of the final solution for the dry reagent for measuring the prothrombin time can be dispensed onto the same. For methods for measuring the prothrombin time using magnetic particles such as this, see, for example, JP H07-135999 A. The entire contents disclosed therein are incorporated herein by reference.

The phrase “undiluted whole blood” used herein refers to whole blood, which is not subjected to any dilution procedure, such as the addition of a dilution buffer, with regard to the whole blood sample after blood sampling. As such, even if the blood is diluted with a citrate solution or other substances contained in the blood-sampling tube at the time of blood sampling (such blood is generally referred to as “citrated whole blood”), so long as the whole blood sample is not subjected to any specific dilution procedure after blood sampling, such blood is within the scope of “undiluted whole blood” as used herein. As such, undiluted whole blood encompasses citrated whole blood and heparinized whole blood that are not subjected to any dilution procedure. The phrase “undiluted plasma” used herein refers to a supernatant obtained by centrifugation of undiluted whole blood, and such plasma is not subjected to a dilution procedure, such as the addition of a dilution buffer. As such, the undiluted plasma encompasses citrated plasma and heparinized plasma that are not subjected to a dilution procedure. Incidentally, the phrase “non-diluted” is synonymous with the term “undiluted” in the present disclosure.

In some embodiments, a dry reagent for measuring the prothrombin time contains tissue thromboplastin. While the amount of tissue thromboplastin to be contained in the dry reagent for measuring the prothrombin time is not particularly limited, for example, the amount of rabbit brain-derived tissue thromboplastin is 1 ng to 1 mg per measurement, and that of rabbit brain-derived phospholipid is 1 ng to 1 mg per measurement.

In some embodiments, the dry reagent for measuring the prothrombin time comprises magnetic particles. Any conventional magnetic particles may be for the dry reagent for measuring the prothrombin time without limitation. Examples of magnetic particles include, but are not limited to, triiron tetraoxide particles, iron sesquioxide particles, iron particles, cobalt particles, nickel particles, and chromium oxide particles. In some embodiments, magnetic particles can be fine particles of triiron tetraoxide. That is, in certain embodiments, fine particles of triiron tetraoxide may be used from the perspective of the intensity of the movement signal of the magnetic particles. The particle diameter of the magnetic particles is not particularly limited, and the average particle diameter can be 0.05 μm to 5 μm, 0.1 μm to 3.0 μm, such as 0.25 μm to 0.5 μm, although not limited thereto. In some embodiments, the average particle diameter of the magnetic particles may be 0.1 m to 3.0 μm. The phrase “average particle diameter” used herein refers to a particle diameter (D50) at a cumulative value of 50% in a particle size distribution by a laser diffraction and scattering method, unless otherwise specified. The magnetic particle content in the dry reagent for measuring the prothrombin time is not particularly limited. For example, such content may be 4 mg/1 mL to 40 mg/1 mL of the final solution.

In some embodiments, the dry reagent for measuring the prothrombin time may further contain, as an optional component, a heparin neutralizer. Any conventional heparin neutralizer may be used as the heparin neutralizer without limitation, and examples thereof include, but are not limited to, polybrene, protamine sulfate, and heparinase. In some embodiments, polybrene may be used as the heparin neutralizer from the perspective of good storage stability and cost effectiveness. The amount of a heparin neutralizer to be incorporated into a dry reagent for measuring the prothrombin time is not particularly limited and may be appropriately determined. When polybrene is used as a heparin neutralizer in some embodiments, the amount of polybrene to be incorporated into the dry reagent for measuring the prothrombin time may be, for example, 50 μg/1 mL to 300 μg/1 mL of the final solution.

In some embodiments, the dry reagent for measuring the prothrombin time comprises a calcium salt. Any conventional calcium salt may be used for the dry reagent without limitation. Examples of inorganic acid calcium salts include calcium chloride, calcium nitrite, calcium sulfate, and calcium carbonate. Examples of organic acid calcium salts include calcium lactate and calcium tartrate. In some embodiments, calcium chloride may be used as the calcium salt. The amount of a calcium salt to be incorporated into a dry reagent for measuring the prothrombin time may appropriately be determined without particular limitation. When a calcium chloride dihydrate is used as the calcium salt, the amount of a calcium chloride dihydrate to be incorporated into the dry reagent for measuring the prothrombin time may be 0.2 μg/1 mL to 2 μg/1 mL of the final solution.

In some embodiments, the dry reagent for measuring the prothrombin time comprises a dry reagent layer solubility improving agent (an agent for improving solubility of the dry reagent layer). Examples of the dry reagent layer solubility improving agent include an amino acid or salt thereof and a saccharide. The amino acid or salt thereof or a saccharide used herein may be any of a neutral amino acid or salt thereof, an acidic amino acid or salt thereof, a basic amino acid or salt thereof, a monosaccharide, and a polysaccharide. Examples of representative acidic amino acids or salts thereof include glutamic acid, sodium glutamate, aspartic acid, and sodium aspartate. Examples of representative neutral amino acids or salts thereof include glycine, glycine hydrochloride, and alanine. Examples of representative basic amino acids or salts thereof include lysine, lysine hydrochloride, and arginine. Examples of monosaccharides include glucose and fructose. Examples of polysaccharides include sucrose, lactose, and dextrin. Among these substances, glycine may be used from the perspective of good solubility of the reagent when a sample is added to the dry reagent for measuring the prothrombin time, good reproducibility of the movement signals of magnetic particles, and good impact resistance. In some other embodiments, specifically, a dry reagent layer solubility improving agent may be glycine.

The amount of the dry reagent layer solubility improving agent to be incorporated into the dry reagent for measuring the prothrombin time, such as the amount of an amino acid or salt thereof or a saccharide, may appropriately be determined without particular limitation. When glycine is used as the dry reagent layer solubility improving agent, in some embodiments, the amount of glycine to be incorporated into the dry reagent for measuring the prothrombin time may be 1.5% by weight or more, 1.6% by weight or more, 1.7% by weight or more, 1.8% by weight or more, 1.9% by weight or more, 2.0% by weight or more, 2.1% by weight or more, 2.2% by weight or more, 2.3% by weight or more, 2.4% by weight or more, 2.5% by weight or more, 2.6% by weight or more, 2.7% by weight or more, 2.8% by weight or more, 2.9% by weight or more, 3.0% by weight or more, 3.1% by weight or more, 3.2% by weight or more, 3.3% by weight or more, 3.4% by weight or more, 3.5% by weight or more, 3.6% by weight or more, 3.7% by weight or more, 3.8% by weight or more, 3.9% by weight or more, 4.0% by weight or more, 4.1% by weight or more, 4.2% by weight or more, 4.3% by weight or more, 4.4% by weight or more, 4.5% by weight or more, 4.6% by weight or more, 4.7% by weight or more, 4.8% by weight or more, or 4.9% by weight or more, for example 5.0% by weight. When glycine is used as the dry reagent layer solubility improving agent, in some embodiments, the amount of glycine to be incorporated into the dry reagent for measuring the prothrombin time may be 5.0% by weight or less, 4.9% by weight or less, 4.8% by weight or less, 4.7% by weight or less, 4.6% by weight or less, 4.5% by weight or less, 4.4% by weight or less, 4.3% by weight or less, 4.2% by weight or less, 4.1% by weight or less, 4.0% by weight or less, 3.9% by weight or less, 3.8% by weight or less, 3.7% by weight or less, 3.6% by weight or less, 3.5% by weight or less, 3.4% by weight or less, 3.3% by weight or less, 3.2% by weight or less, 3.1% by weight or less, 3.0% by weight or less, 2.9% by weight or less, 2.8% by weight or less, 2.7% by weight or less, 2.6% by weight or less, 2.5% by weight or less, 2.4% by weight or less, 2.3% by weight or less, 2.2% by weight or less, 2.1% by weight or less, 2.0% by weight or less, 1.9% by weight or less, 1.8% by weight or less, 1.7% by weight or less, or 1.6% by weight or less, for example 1.5% by weight. In the present disclosure, the amount of glycine to be incorporated into the dry reagent for measuring the prothrombin time encompasses any combination of minimal amount and maximal amount wherein the minimal amount and maximal amount are set to be any of the minimal amounts and the maximal amounts mentioned above. For example, in some embodiments, the amount of glycine to be incorporated into the dry reagent for measuring the prothrombin time may be set as 1.5% to 5.0% by weight, 2.0% to 5.0% by weight, 2.5% to 5.0% by weight, 3.0% to 5.0% by weight, 3.5% to 5.0% by weight, 4.0% to 5.0% by weight, 4.5% to 5.0% by weight, 1.5% to 4.5% by weight, 2.0% to 4.5% by weight, 2.5% to 4.5% by weight, 3.0% to 4.5% by weight, 3.5% to 4.5% by weight, 4.0% to 4.5% by weight, 1.5% to 4.0% by weight, 2.0% to 4.0% by weight, 2.5% to 4.0% by weight, 3.0% to 4.0% by weight, 3.5% to 4.0% by weight, 1.5% to 3.5% by weight, 2.0% to 3.5% by weight, 2.5% to 3.5% by weight, 3.0% to 3.5% by weight, 1.5% to 3.0% by weight, 2.0% to 3.0% by weight, 2.5% to 3.0% by weight, 1.5% to 2.5% by weight, 2.0% to 2.5% by weight, or 1.5% to 2.0% by weight. In some embodiments, when glycine is used as the dry reagent layer solubility improving agent, the amount of glycine to be incorporated into the dry reagent for measuring the prothrombin time may be 1.5% to 4.0% by weight. In other embodiments, when glycine is used as the dry reagent layer solubility improving agent, the amount of glycine to be incorporated into the dry reagent for measuring the prothrombin time may be 2.0% to 3.0% by weight. When glycine is used as the dry reagent layer solubility improving agent for measuring an undiluted plasma sample, the amount of glycine to be incorporated into the dry reagent for measuring the prothrombin time may be within the range mentioned above, such as 1.5% to 4.0% by weight. When glycine is used as the dry reagent layer solubility improving agent for measuring an undiluted whole blood sample, the amount of glycine to be incorporated into the dry reagent for measuring the prothrombin time may be within the range mentioned above, such as 1.5% by weight or more. When glycine is used as the dry reagent layer solubility improving agent for measuring an undiluted whole blood sample, for example, the amount of glycine to be incorporated into the dry reagent for measuring the prothrombin time may be 1.5% to 5.0% by weight, or 1.5% to 4.5% by weight, such as 1.5% to 4.0% by weight. When enabling measurement of both an undiluted plasma sample and an undiluted whole blood sample, when glycine is used as the dry reagent layer solubility improving agent, the amount of glycine to be incorporated into the dry reagent for measuring the prothrombin time may be any combination of these various ranges. It should be noted that the unit “% by weight” used herein indicates the concentration in the final solution; i.e., the final concentration, unless otherwise specified.

In some embodiments, the dry reagent for measuring the prothrombin time comprises a pH buffer (also referred to as a pH adjuster). A buffer supplemented with tissue thromboplastin, magnetic particles, a heparin neutralizer, a calcium salt, and a dry reagent layer solubility improving agent prior to lyophilization is not particularly limited, provided that the buffer exerts buffering actions at pH 6.0 to 8.0. In some embodiments, a pH buffer may be a buffer capable of adjusting the pH level of the reagent to a pH of 6.0 to 8.0, such as about pH 7.35 or about pH 7.5. Examples of buffers that may be used include 40 mM HEPES buffer (pH=7.35) and 40 mM Tris-HCl buffer (pH=7.5).

In some embodiments, the dry reagent for measuring the prothrombin time according to the present disclosure comprises a dry reagent layer reinforcing material (a material for reinforcing the dry reagent layer). Examples of the dry reagent layer reinforcing material include, but are not limited to, bovine serum albumin and human serum albumin. When bovine serum albumin is used as the dry reagent layer reinforcing material, the amount of the dry reagent layer reinforcing material to be incorporated into the dry reagent may be in a range of 0.6 to 2.0 mg/1 ml of the final solution.

In some embodiments, the dry reagent for measuring the prothrombin time according to the present disclosure may comprise, as an optional component, a defoaming agent. Examples of the defoaming agent include, but are not limited to, sorbitan monolaurate, a silicone-based defoaming agent, and a polypropylene glycol-based defoaming agent. When sorbitan monolaurate is used as the defoaming agent, the amount of defoaming agent to be incorporated into the dry reagent for quantification may be in a range of about 0.001% to about 0.010% by weight.

In some embodiments, the method of drying the buffer containing the components described above may be lyophilization from the perspective of solubility of the dry reagent for measuring the prothrombin time, the intensity of the movement signal of magnetic particles, and reproducibility. In some embodiments, the method of drying does not include air-drying.

The method of freezing and lyophilization are not particularly limited. Common techniques of freezing can be employed and, for example, a final solution for the dry reagent for measuring the prothrombin time can be dispensed onto a reaction slide through the dispensing port shown in FIG. 1, and then the reaction slide can be stored and frozen in a freezer maintained at −40° C. or lower for one whole day and night, or and then the reaction slide can be mounted on a lyophilizer in which the shelf temperature is set at −40° C. or lower and stored and frozen therein for one whole day and night, or and then the reaction slide can be frozen instantly with liquid nitrogen. In addition, the technique for lyophilizing the frozen reaction slide is not particularly limited. To exemplify the lyophilizing method, the lyophilizing method includes a method in which the temperature of the frozen reaction slide may be linearly raised from −30° C. to −20° C. over a period of 24 hours in vacuum, the temperature thereof may be linearly raised from −20° C. to 30° C. over a period of 20 hours, lastly the temperature may be maintained at 30° C. for 3 hours, and then dry air may be applied to release the vacuum.

After the lyophilization, the dry reagent for measuring the prothrombin time may be immediately sealed with an aluminum film in a dehumidified environment. While the dehumidified environment is not particularly limited, temperature may be at room temperature of 22° C. to 27° C. and relative humidity may be 35% or lower therein. While specifications of the aluminum film are not particularly limited, a 5-layer structure aluminum film (thickness: 86 μm) comprising a polyester film (thickness: 12 μm), polyethylene resin (thickness: 15 μm), an aluminum foil (thickness: 9 μm), a polyethylene resin (thickness: 20 μm), and a polyethylene film (thickness: 30 μm) adhered with an AC coating agent may be used. The entire dry reagent for measuring the prothrombin time is wrapped with the aluminum foil and sealed via heat adhesion. The dry reagent for measuring the prothrombin time may be refrigerated in a sealed state before using the same for measuring the prothrombin time.

The dry reagent for measuring the activated partial thromboplastin time of the present disclosure can be prepared with the addition of a phospholipid and an activator instead of tissue thromboplastin among the components of the dry reagent for measuring the prothrombin time described above. Various naturally-occurring or artificially synthesized phospholipids can be used. Examples of naturally-occurring phospholipids include rabbit brain-derived cephalin, pig-derived lipid, chicken-derived lipid, and soybean-derived lipid. Examples of artificially synthesized phospholipids that can be used include phosphatidylcholine, phosphatidylserine, phosphatidylethanolamine, and phosphatidylinositol. Examples of activators that can be used include inorganic matter such as silica and kaolin and organic matter such as ellagic acid and actin.

### EXAMPLES

The present disclosure has been described in general terms above. However, the present disclosure can be further understood with reference to the specific examples below. It should be noted that the examples presented here are provided solely for illustrative purposes and do not limit the scope of the present invention.

### Example 1: Discrimination of Samples not Receiving Administration of Direct Oral Anticoagulant from Samples Receiving Administration of Edoxaban Using Dry PT Reagent

A lyophilized product containing rabbit brain-derived tissue thromboplastin and rabbit brain-derived phospholipid (Thermo Fisher Scientific) was dissolved in 10 ml of purified water to obtain a tissue thromboplastin solution. The tissue thromboplastin solution was centrifuged at 4,000 rpm and 4° C. for 30 minutes, and the supernatant was removed to obtain pellets of rabbit brain-derived tissue thromboplastin. A 40 mM HEPES buffer (pH 7.35) supplemented with 10 mM CaCl₂·2H₂O, 2.5 (wt/v) % glycine, 3.0 mg/ml bovine serum albumin, 0.005 (wt/v) % sorbitan monolaurate, and 150 g/ml GPRP-amide was added to the pellets of rabbit brain-derived tissue thromboplastin and dispersed therein with stirring to obtain a reagent solution for measuring the prothrombin time. To 35 ml of the reagent solution, 0.47 g of triiron tetraoxide (product name: AAT-03; average particle diameter: 0.35 m; Toda Kogyo Corp.) was added and suspended to obtain a final solution. The final solution (25 μl) was dispensed onto the reaction slide shown in FIG. 1. The reaction slide was stored and frozen in a freezer maintained at −40° C. for one whole day and night. Subsequently, the frozen reaction slide was lyophilized. Lyophilization was performed under the conditions in which the temperature was linearly raised from −30° C. to −20° C. over a period of 24 hours in vacuum, the temperature was linearly raised from −20° C. to 30° C. over a period of 20 hours, the temperature was maintained at 30° C. for 3 hours, and dry air was then applied to release the vacuum. The lyophilized reagent was immediately sealed with an aluminum film in a dehumidified environment.

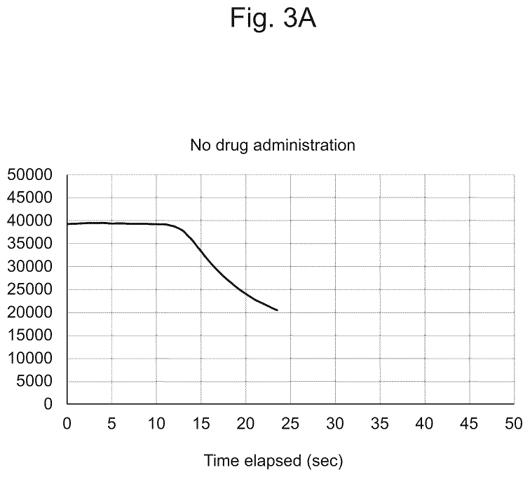

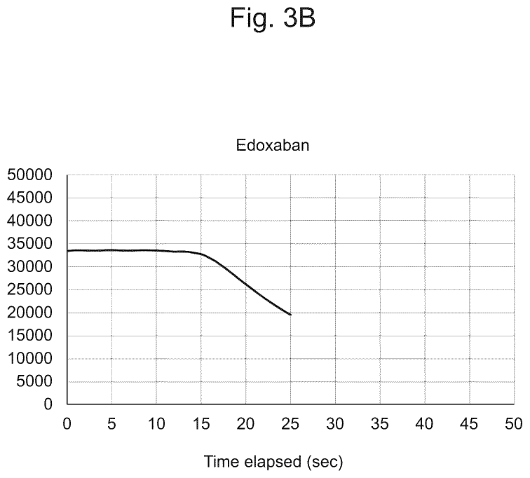

The samples not receiving administration of direct oral anticoagulants were discriminated from the samples receiving administration of edoxaban using dry PT reagents in the manner described below. At the outset, 26 samples not receiving administration of direct oral anticoagulants (including 11 samples receiving administration of warfarin, 2 samples receiving administration of heparin, and 3 samples not receiving administration of anticoagulants) and 37 samples receiving administration of edoxaban were prepared. Subsequently, the lyophilized reagent was mounted on the blood coagulation analyzer CG02N (A&T Corporation), and 25 μl of the prepared samples were added to determine the clotting time and PT-INR of each sample. The samples exhibiting PT-INR of lower than 2.0 were excluded as the samples without coagulation abnormalities. For the 17 samples not receiving administration of direct oral anticoagulants and the 34 samples receiving administration of edoxaban that were not excluded, the clot waveform data obtained when the samples were analyzed using CG02N were obtained. On the basis of the clot waveform data obtained, (1) the maximal value of the movement level of the magnetic particles, (2) the time at which the maximal value of the movement level of the magnetic particles was obtained, (3) the movement level of the magnetic particles when the movement level of the magnetic particles was attenuated by 5% relative to the maximal level, and (4) the time at which the movement level of the magnetic particles was attenuated by 5% relative to the maximal level were determined as feature amounts of the clot waveform. On the basis of feature amounts (1) to (4), the inclination of the clot waveform between “the maximal value of the movement level of the magnetic particles and the movement level of the magnetic particles attenuated by 5% relative to the maximal value of the movement level of the magnetic particles was obtained. FIGS. 3A and 3B each show the clot waveform of the samples not receiving administration of direct oral anticoagulants and that of the samples receiving administration of edoxaban.

Table 1 shows the means and the standard errors of the inclinations of the clot waveforms of the samples not receiving administration of direct oral anticoagulants and of the samples receiving administration of edoxaban. The means of the inclinations of the clot waveforms of the samples not receiving administration of direct oral anticoagulants and of the samples receiving administration of edoxaban were −1219.8 and −664.0, respectively. The results demonstrate that the inclination of the samples receiving administration of edoxaban is larger than that of the samples not receiving administration of direct oral anticoagulants.

TABLE 1

| | | Samples not | Samples |
|---|---|---|---|
| | | receiving | receiving |
| | | administration | administration |
| | | of DOAC | of edoxaban |
| Slope of | Mean | −1219.8 | −664.0 |
| clot | Standard error | 152.9 | 344.6 |
| waveform | | | |

Table 2 shows the cross tabulation showing the results of determination performed using an algorithm which determine samples exhibiting the inclination of the clot waveform of −1000 or larger as the samples that have received administration of edoxaban, and samples exhibiting the inclination of smaller than −1000 as the samples that have not received administration of direct oral anticoagulants, for discrimination of the samples not receiving administration of direct oral anticoagulants from the samples receiving administration of edoxaban.

TABLE 2

| | | Sample type | | |
|---|---|---|---|---|
| | | Samples | Samples not | |
| | | receiving | receiving | |
| | | administration | administration | |
| | | of edoxaban | of DOAC | Total |
| Determination | With | 29 | 1 | 30 |
| by inclination of | edoxaban | | | |
| clot waveform | Without | 5 | 16 | 21 |
| | edoxaban | | | |

| | | | |
|---|---|---|---|
| Total | 34 | 17 | 51 |

The sensitivity of determination as to the administration of edoxaban and the specificity thereof calculated based on the table above were 0.85 and 0.94, respectively. This indicates that determination as to the administration of edoxaban based on the inclination of the clot waveform can be performed with high sensitivity and high specificity.

### Example 2: Discrimination of Samples not Receiving Administration of Direct Oral Anticoagulants from Samples Receiving Administration of Rivaroxaban Using Dry PT Reagent

The samples not receiving administration of direct oral anticoagulants were discriminated from the samples receiving administration of rivaroxaban using the lyophilized reagent prepared in Example 1 as the dry PT reagent in the manner described below.

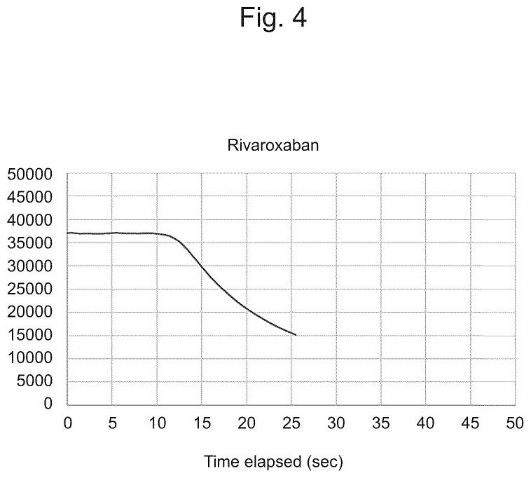

At the outset, 26 samples not receiving administration of direct oral anticoagulants (including 11 samples receiving administration of warfarin, 2 samples receiving administration of heparin, and 3 samples not receiving administration of anticoagulants) and 24 samples receiving administration of rivaroxaban were prepared. Subsequently, the lyophilized reagent was mounted on the blood coagulation analyzer CG02N (A&T Corporation), and 25 μl of the prepared samples were added to determine the clotting time and PT-INR of each sample. Samples exhibiting PT-INR of lower than 2.0 were excluded as the samples without coagulation abnormalities. For the 17 samples not receiving administration of direct oral anticoagulants and the 22 samples receiving administration of rivaroxaban that were not excluded, the clot waveform data obtained when the samples were analyzed using CG02N were obtained. On the basis of the clot waveform data obtained, in addition, (1) the maximal value of the movement level of the magnetic particles, (2) the time at which the maximal value of the movement level of the magnetic particles was obtained, (3) the movement level of the magnetic particles when the movement level of the magnetic particles was attenuated by 5% relative to the maximal level, and (4) the time at which the movement level of the magnetic particles was attenuated by 5% relative to the maximal level were determined as feature amounts of the clot waveform. On the basis of feature amounts (1) to (4), the inclination of the clot waveform from the maximal value of the movement level of the magnetic particles to the movement level of the magnetic particles attenuated by 5% relative to the maximal value of the movement level of the magnetic particles was obtained. FIG. 4 shows the clot waveform of the samples receiving administration of rivaroxaban.

Table 3 shows the means and the standard errors of the inclinations of the clot waveforms of the samples not receiving administration of direct oral anticoagulants and of the samples receiving administration of rivaroxaban. The means of the inclinations of the clot waveforms of the samples not receiving administration of direct oral anticoagulants and of the samples receiving administration of rivaroxaban were −1219.8 and −805.9, respectively. The results demonstrate that the inclination of the samples receiving administration of rivaroxaban is larger than that of the samples not receiving administration of direct oral anticoagulants.

TABLE 3

| | | Samples not | Samples |
|---|---|---|---|
| | | receiving | receiving |
| | | administration | administration |
| | | of DOAC | of rivaroxaban |
| Slope of clot | Mean | −1219.8 | −805.9 |
| waveform | Standard error | 152.9 | 183.0 |

Table 4 shows the cross tabulation showing the results of determination performed using an algorithm which determine samples exhibiting the inclination of the clot waveform of −1000 or larger as the samples receiving administration of rivaroxaban and samples exhibiting the inclination of smaller than −1000 as the samples not receiving administration of direct oral anticoagulants, for discrimination of the samples not receiving administration of direct oral anticoagulants from the samples receiving administration of rivaroxaban.

TABLE 4

| | | Sample type | | |
|---|---|---|---|---|
| | | Samples | Samples not | |
| | | receiving | receiving | |
| | | administration | administration | |
| | | of rivaroxaban | of DOAC | Total |
| Determination | With | 19 | 1 | 20 |
| by inclination of | rivaroxaban | | | |
| clot waveform | Without | 3 | 16 | 19 |
| | rivaroxaban | | | |

| | | | |
|---|---|---|---|
| Total | 22 | 17 | 39 |

The sensitivity of determination as to the administration of rivaroxaban and the specificity thereof calculated based on the table above were 0.86 and 0.94, respectively. This indicates that determination as to the administration of rivaroxaban based on the inclination of the clot waveform can be performed with high sensitivity and high specificity.

### Example 3: Discrimination of Dabigatran Administration Using Dry APTT Reagent

A 60 mM HEPES buffer (pH 7.35, 20 ml) supplemented with 4.0 (wt/v) % glycine, 20 mM CaCl₂·2H₂O, and 6.0 mg/ml bovine serum albumin was added to 20 ml of rabbit brain-derived cephalin and dispersed therein with stirring to obtain an activated partial thromboplastin reagent. To 35 ml of the reagent solution, 1.41 g of triiron tetraoxide (product name: AAT-03; average particle diameter: 0.35 μm; Toda Kogyo Corp.) was added and suspended to obtain a final solution. The final solution (25 μl) was dispensed onto the reaction slide shown in FIG. 1. The reaction slide was stored and frozen in a freezer maintained at −40° C. for one whole day and night. Subsequently, the frozen reaction slide was lyophilized. Lyophilization was performed under the conditions in which the temperature was linearly raised from −30° C. to −20° C. over a period of 24 hours in vacuum, the temperature was linearly raised from −20° C. to 30° C. over a period of 20 hours, the temperature was maintained at 30° C. for 3 hours, and dry air was then applied to release the vacuum. The lyophilized reagent was immediately sealed with an aluminum film in a dehumidified environment.

The samples receiving administration of dabigatran were examined using the dry APTT reagent in the manner described below. DMSO was added to 10 mg of dabigatran to bring the total amount of the solution to 10 ml, and a solution of 1,000 μg/ml of dabigatran was prepared. The dabigatran solution was diluted with physiological saline to prepare serial dilution samples of 0, 0.9, 1.8, 5, and 10 g/ml dabigatran. Each of the serial dilution samples of dabigatran were added in an amount of 100 μl each to 900 μl of normal plasma to prepare plasma samples supplemented with 0, 90, 180, 500, and 1,000 ng/ml of dabigatran. The lyophilized reagent was mounted on the blood coagulation analyzer CG02N (A&T Corporation), and 25 μl of the prepared samples were added to determine the clotting time of each sample.

Table 5 shows the clotting time of dabigatran-supplemented plasma. The lyophilized reagent was found to prolong the clotting time depending on the concentration of dabigatran.

TABLE 5

| | Dabigatran concentration in plasma (ng/mL) | | | | |
|---|---|---|---|---|---|
| | 0 | 90 | 180 | 500 | 1,000 |
| Mean clotting time (sec) | 29.0 | 51.1 | 60.8 | 86.2 | 119.0 |

### INDUSTRIAL APPLICABILITY

The present disclosure allows a rapid determination as to whether or not a patient has received administration of an anticoagulant, such as DOAC. This allows a rapid determination as to the administration of an anticoagulant, such as DOAC, to a patient whose drug history is unknown in an emergency medical setting, such as critical care. By determining DOAC administration, appropriate hemostasis strategies can be developed. By determining DOAC administration, further, a survival rate of a patient receiving administration of DOAC can be improved in the event of bleeding. By determining DOAC administration, furthermore, the period up to hemostasis can be shortened to decrease the amount of the blood preparations to be used.

All publications, patents, and patent applications cited herein are incorporated herein by reference in their entirety.

### DESCRIPTION OF SYMBOLS

A: Transparent resin plate
B: Transparent resin plate
C: White resin plate
D: Reagent filling unit

1. A test method for determining whether or not a direct oral anticoagulant (DOAC) has been administered to a patient, comprising:
(i) adding a sample to a dry reagent containing magnetic particles for measuring prothrombin time or a dry reagent containing magnetic particles for measuring activated partial thromboplastin time;
(ii) allowing the magnetic particles in the reagent to move after the addition of the sample, and monitoring the movement signal of the magnetic particles;
(iii) analyzing the movement signal of the magnetic particles monitored in step (ii); and
(iv) comparing the results of analysis performed in step (iii) with a threshold that enables a clear distinction between control 1 not receiving administration of DOAC (“no-DOAC control 1”) and control 2 receiving administration of DOAC (“DOAC control 2”).

2. The method according to claim 1, wherein the threshold is the arithmetic mean of the inclination of the change in the movement signal of the magnetic particles of no-DOAC control 1 over time and the inclination of the change in the movement signal of the magnetic particles of DOAC control 2 over time, wherein the inclination of the change in the movement signal of the magnetic particles over time is the inclination of the clot waveform, which is determined by subtracting the movement level of the magnetic particles attenuated by a given percentage, selected from the group consisting of 3%, 4%, 5%, 6%, 7%, 8%, 9%, 10%, 11%, 12%, 13%, 14%, and 15%, relative to the maximal value of the movement level of the magnetic particles, from the maximal value of the movement level of the magnetic particles.

3. The method according to claim 1, wherein the threshold is the arithmetic mean of the minimal value of the differential value of the inclination of the change in the movement signal of the magnetic particles of no-DOAC control 1 over time and the minimal value of the differential value of the inclination of the change in the movement signal of the magnetic particles of DOAC control 2 over time, wherein the inclination of the change in the movement signal of the magnetic particles over time is the inclination of the clot waveform, which is determined by subtracting the movement level of the magnetic particles attenuated by a given percentage, selected from the group consisting of 3%, 4%, 5%, 6%, 7%, 8%, 9%, 10%, 11%, 12%, 13%, 14%, and 15%, relative to the maximal value of the movement level of the magnetic particles, from the maximal value of the movement level of the magnetic particles.

4. The method according to claim 1, wherein the threshold is the arithmetic mean of the minimal value of the second-order differential value of the inclination of the change in the movement signal of the magnetic particles of no-DOAC control 1 over time and the minimal value of the second-order differential value of the inclination of the change in the movement signal of the magnetic particles of DOAC control 2 over time, wherein the inclination of the change in the movement signal of the magnetic particles over time is the inclination of the clot waveform, which is determined by subtracting the movement level of the magnetic particles attenuated by a given percentage, selected from the group consisting of 3%, 4%, 5%, 6%, 7%, 8%, 9%, 10%, 11%, 12%, 13%, 14%, and 15%, relative to the maximal value of the movement level of the magnetic particles, from the maximal value of the movement level of the magnetic particles.

5. The method according to claim 1, wherein step (iv) comprises:
(iv-1) comparing the results of analysis performed in step (iii) with a first threshold that enables a clear distinction between no-DOAC control 1 and DOAC control 2; and
(iv-2) comparing the results of analysis performed in step (iii) with a second threshold that enables a clear distinction between control 3 receiving administration of a non-DOAC anticoagulant (“non-DOAC anticoagulant control 3”) and DOAC control 2.

6. The method according to claim 5, wherein the threshold is the arithmetic mean of the inclination of the change in the movement signal of the magnetic particles of non-DOAC anticoagulant control 3 over time and the inclination of the change in the movement signal of the magnetic particles of DOAC control 2 over time, wherein the inclination of the change in the movement signal of the magnetic particles over time is the inclination of the clot waveform, which is determined by subtracting the movement level of the magnetic particles attenuated by a given percentage, selected from the group consisting of 3%, 4%, 5%, 6%, 7%, 8%, 9%, 10%, 11%, 12%, 13%, 14%, and 15%, relative to the maximal value of the movement level of the magnetic particles, from the maximal value of the movement level of the magnetic particles.

7. The method according to claim 5, wherein the threshold is the arithmetic mean of the minimal value of the differential value of the inclination of the change in the movement signal of the magnetic particles of non-DOAC anticoagulant control 3 over time and the minimal value of the differential value of the inclination of the change in the movement signal of the magnetic particles of DOAC control 2 over time, wherein the inclination of the change in the movement signal of the magnetic particles over time is the inclination of the clot waveform, which is determined by subtracting the movement level of the magnetic particles attenuated by a given percentage, selected from the group consisting of 3%, 4%, 5%, 6%, 7%, 8%, 9%, 10%, 11%, 12%, 13%, 14%, and 15%, relative to the maximal value of the movement level of the magnetic particles, from the maximal value of the movement level of the magnetic particles.

8. The method according to claim 5, wherein the threshold is the arithmetic mean of the minimal value of the second-order differential value of the inclination of the change in the movement signal of the magnetic particles of non-DOAC anticoagulant control 3 over time and the minimal value of the second-order differential value of the inclination of the change in the movement signal of the magnetic particles of DOAC control 2 over time, wherein the inclination of the change in the movement signal of the magnetic particles over time is the inclination of the clot waveform, which is determined by subtracting the movement level of the magnetic particles attenuated by a given percentage, selected from the group consisting of 3%, 4%, 5%, 6%, 7%, 8%, 9%, 10%, 11%, 12%, 13%, 14%, and 15%, relative to the maximal value of the movement level of the magnetic particles, from the maximal value of the movement level of the magnetic particles.

9. The method according to claim 1, wherein step (iv) comprises:
(iv-1) comparing the results of analysis performed in step (iii) with a first threshold that enables a clear distinction between no-DOAC control 1 and DOAC control 2; and
(iv-2) comparing the results of analysis performed in step (iii) with a third threshold that enables a clear distinction between control 4 that has not received administration of an anticoagulant (“no-anticoagulant control 4”) and DOAC control 2.

10. The method according to claim 9, wherein the threshold is the arithmetic mean of the inclination of the change in the movement signal of the magnetic particles of no-anticoagulant control 4 over time and the inclination of the change in the movement signal of the magnetic particles of DOAC control 2 over time, wherein the inclination of the change in the movement signal of the magnetic particles over time is the inclination of the clot waveform, which is determined by subtracting the movement level of the magnetic particles attenuated by a given percentage, selected from the group consisting of 3%, 4%, 5%, 6%, 7%, 8%, 9%, 10%, 11%, 12%, 13%, 14%, and 15%, relative to the maximal value of the movement level of the magnetic particles, from the maximal value of the movement level of the magnetic particles.

11. The method according to claim 9, wherein the threshold is the arithmetic mean of the minimal value of the differential value of the inclination of the change in the movement signal of the magnetic particles of no-anticoagulant control 4 over time and the minimal value of the differential value of the inclination of the change in the movement signal of the magnetic particles of DOAC control 2 over time, wherein the inclination of the change in the movement signal of the magnetic particles over time is the inclination of the clot waveform, which is determined by subtracting the movement level of the magnetic particles attenuated by a given percentage, selected from the group consisting of 3%, 4%, 5%, 6%, 7%, 8%, 9%, 10%, 11%, 12%, 13%, 14%, and 15%, relative to the maximal value of the movement level of the magnetic particles, from the maximal value of the movement level of the magnetic particles.

12. The method according to claim 9, wherein the threshold is the arithmetic mean of the minimal value of the second-order differential value of the inclination of the change in the movement signal of the magnetic particles of no-anticoagulant control 4 over time and the minimal value of the second-order differential value of the inclination of the change in the movement signal of the magnetic particles of DOAC control 2 over time, wherein the inclination of the change in the movement signal of the magnetic particles over time is the inclination of the clot waveform, which is determined by subtracting the movement level of the magnetic particles attenuated by a given percentage, selected from the group consisting of 3%, 4%, 5%, 6%, 7%, 8%, 9%, 10%, 11%, 12%, 13%, 14%, and 15%, relative to the maximal value of the movement level of the magnetic particles, from the maximal value of the movement level of the magnetic particles.

13. The method according to claim 1, wherein the threshold is a threshold that makes the inclination in the clot waveform reach −1000, wherein the inclination is determined by subtracting the movement level of the magnetic particles attenuated by 5%, relative to the maximal value of the movement level of the magnetic particles, from the maximal value of the movement level of the magnetic particles, when analysis is performed using a CG02N analyzer and using Drihemato PT reagent as a dry reagent containing magnetic particles for measuring the prothrombin time and when Drihemato PT reagent is supplemented with 25 μl of a citrated whole blood sample, or a threshold that corresponds thereto when analysis is performed using another apparatus and using another dry reagent.

14. The method according to claim 13, further comprising determining that the sample is derived from a patient not receiving administration of DOAC when the inclination of the change in the movement signal of the magnetic particles over time is higher (<−1000) than the threshold (−1000) or determining that a sample is derived from a patient receiving administration of DOAC when the inclination of the change in the movement signal of the magnetic particles over time is the threshold (−1000) or lower (>−1000) than the threshold (−1000),
wherein the inclination of the change in the movement signal of the magnetic particles over time is the inclination of the clot waveform, which is determined by subtracting the movement level of the magnetic particles attenuated by 5%, relative to the maximal value of the movement level of the magnetic particles, from the maximal value of the movement level of the magnetic particles.

15. The method according to claim 13, wherein, when the sample is determined to be derived from a patient receiving administration of DOAC as a result of comparison with the threshold, the method further comprises:
administering a reversal agent for DOAC or suggesting administration of a reversal agent for DOAC to the patient.

16. The method according to claim 1, wherein DOAC is selected from the group consisting of dabigatran, rivaroxaban, apixaban, edoxaban, and betrixaban.

17. A program for executing the method of claim 1.

18. An information recording medium comprising the program of claim 17 recorded thereon.

19. An apparatus for testing an anticoagulant comprising the program of claim 17 integrated therein or the information recording medium comprising the program.

20. The method of claim 1, wherein the analyzing is carried out by an apparatus.
